(19) United States
(12) Patent Application Publication
(10) Pub. No.: US 2003/0158093 A1
(43) Pub. Date: Aug. 21, 2003

(54) Bifunctional glycopeptide antibiotics and combinatorial libararies thereof

(76) Inventors: Binyuan Sun, Princeton, NJ (US); Zhong Chen, Princeton, NJ (US); Daniel Kahne, Princeton, NJ (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/223,569

(22) Filed: Aug. 19, 2002

Related U.S. Application Data
(60) Provisional application No. 60/313,271, filed on Aug. 17, 2001.

(51) Int. Cl. A61K 38/14; C07K 9/00

(52) U.S. Cl. 514/8; 530/322

(57) ABSTRACT

The present invention relates to a the design of a large class of antibiotics comprised of aglycones of vancomycin or derivatives of an aglycone of vancomycin attached to the anomeric site of moenomycin or a moenomycin derivative.

### CROSS-REFERENCE TO RELATED APPLICATIONS

This application claims benefit of provisional application serial No. 60/313,271, filed Aug. 17, 2001, the disclosure of which is hereby incorporated by reference in its entirety.

### FIELD OF THE INVENTION

The present inventions relate to the design of a large class of new antibiotics comprised of an aglycone of vancomycin or a derivative of the aglycone of vancomycin attached to the anomeric carbon of moenomycin or a moenomycin derivative.

### BACKGROUND OF THE INVENTION

The emergence of resistance to vancomycin in enterococcal strains has aroused considerable concern. See e.g., Walsh, C. T., Nature 2000, 406 775. Efforts to overcome resistance have led to the development of a new class of vancomycin derivatives containing hydrophobic substituents on the vancosamine sugar. Nagaraj an, R., J. Antibiot. 1993, 46, 118. These glycolipid derivatives are more active than vancomycin against both sensitive and resistant enterococcal strains. It is possible that these glycolipid derivatives of vancomycin are bifunctional molecules, consisting of two biologically active components that interact with different cellular targets. See Ge, M. et al., Science, 1999, 284, 507; Kerns, R. et al., J. Am. Chem. Soc., 2000, 122, 12608. The aglycone binds to the D-Ala-D-Ala dipeptide terminus of peptidoglycan precursors and the substituted disaccharide interacts with proteins involved in the transglycosylation step of peptidoglycan synthesis. This is one possible mechanism for how the compounds overcome resistance. Other mechanisms have been proposed and tested. See e.g., Williams, D. H. et al., Angew. Chem. Int. Ed., 1999, 38, 1172; Rao, J. et al., Science, 1998, 280, 708; Sundram, U. N. et al., J. Am. Chem. Soc., 1996, 118, 13107; Nicolaou, K. C. et al., Angew. Chem. Int. Ed., 2000, 39, 3823.

Certain substituent changes to the disaccharide of vancomycin have been explored, but previous efforts have resulted in limited changes to the sugars attached to the vancomycin aglycone. Both chemical and enzymatic methods have been used to make a limited number of vancomycin derivatives. See e.g., Ge, M. et al., J. Am. Chem. Soc., 1998, 120, 11014; Thompson, C. et al., J. Am. Chem. Soc., 1999, 121, 1237; Nicolaou, K. C. et al., Angew. Chem. Int. Ed., 1999, 38, 240; Solenberg, P. J. et al., Chemistry & Biology, 1997, 4, 195; Losey, H. C. et al., Biochemistry, 2001, 40, 4745. It would be useful to have an efficient, general strategy to attach a wide variety of different sugars to the vancomycin aglycone. If glycolipid derivatives of vancomycin were bifunctional, then the glycosidic linkage to the phenol might not be critical. If not, then simpler linkers might be substituted, which would permit rapid exploration of a wide range of different carbohydrate moieties. The present inventions relate to the design of a large class of new antibiotics comprised of hosts that bind to cell surface peptides attached to specific inhibitors for peptidoglycan-processing enzymes.

### SUMMARY OF THE INVENTION

The present inventions are directed to compounds having antibiotic activity comprising a polypeptide attached to the anomeric carbon of a saccharide. In certain embodiments, the saccharide can be moenomycin or a moenomycin derivative and the polypeptide an aglycone of vancomycin. The saccharide and polypeptide can be attached erg., by a coupling moiety. The present inventions also comprise combinatorial libraries of a plurality of moenomycin saccharide derivatives bonded to at least one aglycone of vancomycin. The present invention has a variety of research, clinical and therapeutic applications. In light of the emergence of resistance to vancomycin, there is a need for identifying additional antibacterial compounds. In addition, the large family of compounds of the present invention may be administered to treat bacterial infections in an animal, for example, humans.

The present invention is generally directed to compounds comprising a polypeptide attached to the anomeric carbon of a saccharide. The saccharide can be, preferably, moenomycin or a moenomycin derivative, while the polypeptide can be an aglycone of vancomycin. “Aglycone of vancomycin” refers to an aglycone of vancomycin or a derivative of aglycone. Derivatives of an aglycone of vancomycin are known in the art and examples include desleucyl vancomycin aglycone, or N- or C-terminal modified vancomycin aglycone.

In certain embodiments, the saccharide can be moenomycin or a moenomycin derivative, such as, for example, a disaccharide derivative of moenomycin, a trisaccharide derivative of moenomycin, or a functionalized disaccharide derivative based on moenomycin, e.g., compound 12. In some instances, the anomeric carbon of the saccharide was previously occupied by a phospholipid. The peptide may be attached to the reducing end of a carbohydrate either directly or indirectly, such as by a glycosidic coupling moiety. Examples of such coupling moieties include, but are not limited to, alkanes, alkenes, alkynes, polyamines, ethanolamines, spermine, spermadine, amides, polyamides, carbonyl-containing moieties, saturated carbon atoms, heteroatoms, aromatic spacers attached to one or more unsaturated carbon to carbon bonds. The coupling moieties also include one or more ethylene glycol units, formed, for example, by using reagents such as chloroethanol.

Compound 5 is a disaccharide linked to a vancomycin aglycone without an anomeric phospholipid. Compound 5 is significantly more active against resistant strains than compound 2, a derivative of vancomycin. Compound 5 also demonstrated activity against resistant strains that was comparable to or better than compound 1b, which is a vancomycin derivative containing hydrophobic substituents on the vancosamine sugar and a natural glycosidic linkage. Compound 5 maintained excellent activity against sensitive strains. Compound 5 was also more active than compound 4 even though it lacked the phospholipid anchor, which previously had been suggested as critical for biological activity. See El-Abadla, N. et al., Tetrahedron, 1999, 55, 699; Sofia, M. et al., J. Med. Chem., 1999, 42, 3193.

The present inventions also include combinatorial libraries comprising a plurality of moenomycin saccharide derivatives bonded to at least one aglycone of vancomycin. In some embodiments, the phospholipid of the moenomycin derivative has been removed. The aglycone derivative of vancomycin can be, for example, desleucyl vancomycin aglycone, or N- or C- terminal modified vancomycin aglycone and the moenomycin saccharide derivative can be a trisaccharide derivative of moenomycin, or a functionalized disaccharide derivative based on moenomycin, e.g., compound 12. The inventions also include compositions comprising the claimed compounds, and in some embodiments, further includes a pharmaceutically acceptable salt.

### EXAMPLES

In order that the invention disclosed herein may be more efficiently understood, examples are provided below. It should be understood that these examples are for illustrative purposes only and are not to be construed as limiting the invention in any manner.

### Example 1

Preparation of Vancomycin Derivatives

To evaluate the importance of the glycosidic linkage in the activity of glycolipid derivatives of vancomycin (compound 1a), compound 2 was prepared by the route shown in Scheme 1.

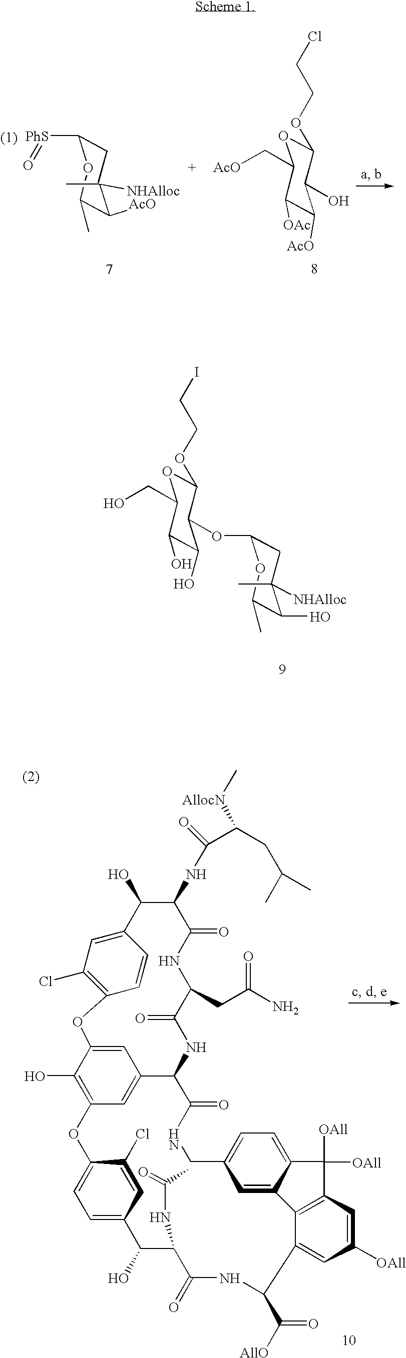

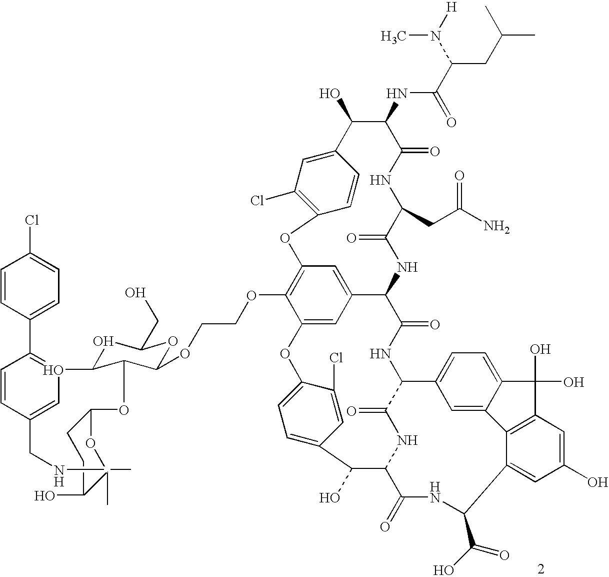

Briefly, compound 7 and compound 10 were synthesized by utilizing procedures described, for example, in Thompson, C. et al., J. Am. Chem. Soc., 1999, 121, 1237. Compound 8 was prepared from tri-O-acetyl-D-glucal by a one-step procedure described by Chen, Z. Ph.D. Dissertation, Princeton University, Princeton, N.J. 2001. The compound was found to have excellent activity against sensitive strains as demonstrated by the data in Table 1 set forth below, wherein MIC is defined as the lowest antibiotics concentration that resulted in visible growth after incubation at 35° C. for 22h.

TABLE 1
MICs of Vancomycin Derivatives*

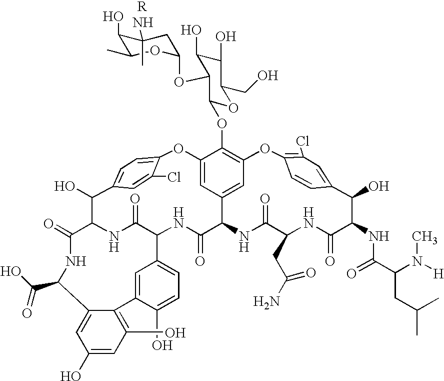

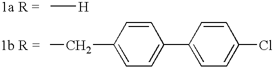

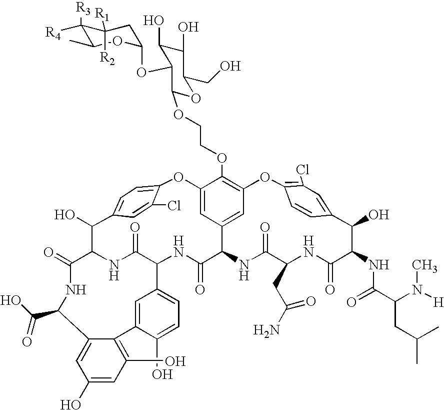

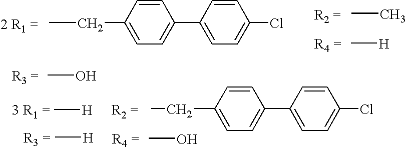

| | |
|---|---|
| E. faecium | E. faecalis |

| | | resistant | | resistant | |
|---|---|---|---|---|---|
| | sensitive | (VanA) | sensitive | (VanB) | S. aureus |
| 1a | 2 | >500 | 16 | >500 | 4 |
| 1b | <0.025 | 12.5 | 0.1 | 12.5 | <0.025 |
| 2 | <0.01 | 63 | 0.05 | 32 | 0.2 |
| 3 | <0.1 | 125 | 0.25 | 250 | 2 |

^{a}MIC values (μg/ml) were obtained using a standard microdilution assay. Bacterial strains used: ^{b}49624; ^{c}CL4931; ^{d}29212; ^{e}CL4877; ^{f}29213.

When compared with compound 1b, which contains the natural glycosidic linkage, compound 2 shows a decrease in activity (2-5 fold) against resistant strains, indicating that the structure of the linker has an effect.

The present inventions also are directed to improvements of the activity of the linked compound 2. Solid phase methods have been developed to make substituted disaccharide libraries containing hundreds to thousands of members. See Liang, R. et al., Science, 1996, 274, 1520. Conservative changes to the natural disaccharide were explored prior to synthesizing a carbohydrate library to determine whether changing the carbohydrate structure could lead to significant improvements in activity. A few conservative changes were made to the natural disaccharide, but each of them led to a decrease in activity. For example, compound3 in Table 1, an isomer of compound 2, is less active against both sensitive and resistant strains as compared to compound 1b or compound 2. Compound 3 was synthesized from compound 8 and 3-azido-4-O-acetyl-1-phenylsulfinyl-2, 3, 6-trideoxy-β-L-ribo-hexopyranoside by a similar procedure to that described in Scheme 1. The difference in activity between compound 2 and compound 3 suggests that the substituted disaccharide may interact with a specific cellular target.

The chlorobiphenyl disaccharide moiety on vancomycin analogs (compounds 1b and 2) may overcome resistance by interacting with proteins involved in the transglycosylation step of cell wall biosynthesis. See Ge, M. et al., Science, 1999, 284, 507; Kems, R. et al., J. Am. Chem. Soc., 2000, 122, 12608; Goldman et al., Microbiol. Lett., 2000, 183, 209. “Transglycosylation” as used herein refers to a process catalyzed by enzymes involved in the final stages of cell wall biosynthesis. This may explain the activity of compound 1b and compound 2 against resistant bacterial strains. Replacing the disaccharide on compound2 with a known transglycosylase inhibitor may produce a still more active compound. One very effective transglycosylase inhibitor, moenomycin, is a glycophospholipid containing five hexoses. See El-Abadla, N. et al., Tetrahedron, 1999, 55, 699. Solid phase libraries of disaccharides based on a fragment of moenomycin have been made. Compound 4 has been identified as having both good antibacterial activity and an ability to inhibit transglycosylation. See El-Abadla, N. et al., Tetrahedron, 1999, 55, 699; Sofia, M. et al., J. Med. Chem., 1999, 42, 3193; Goldman, R. C. et al. Bioorg. Med. Chem. Lett., 2000, 10, 2251; Baizman, E. R. et al., Microbiology, 2000, 146, 3129. This disaccharide is a better starting point than the vancomycin disaccharide because of its improved transglycosylase inhibitory activity. The disaccharide without the anomeric phospholipid was prepared, as presented in Scheme 2, and linked it to the vancomycin aglycone to make compound 5.

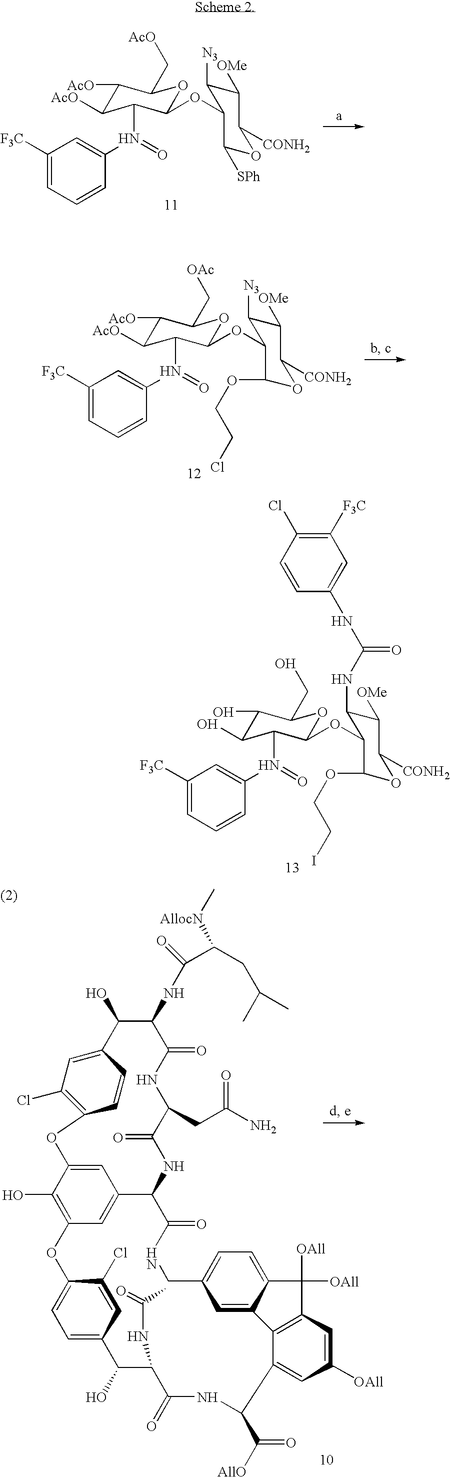

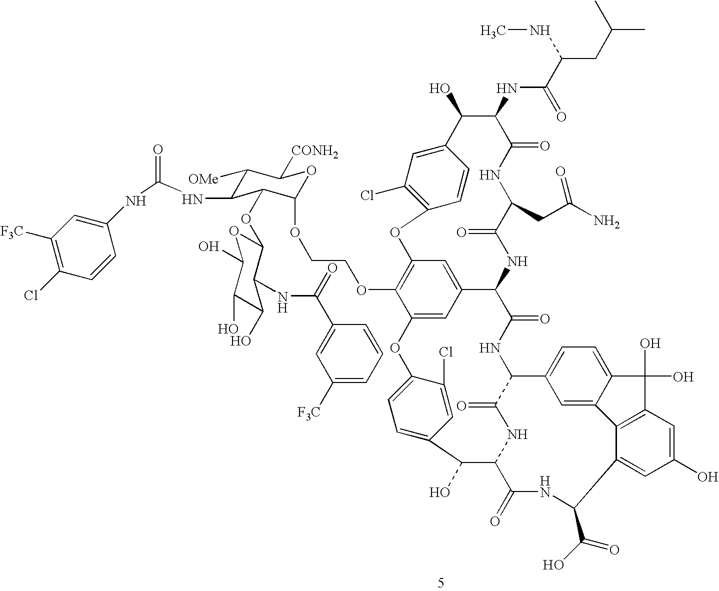

Compound 11 was prepared according to the procedure disclosed in Sofia, M. et al., J. Med. Chem., 1999, 42, 3193. The activities of compound 4, compound 5 and a related analog that lacks a phospholipid, compound 6, are presented in Table 2 below.

TABLE 2
MICs of Moenomycin Disaccharide Derivatives**

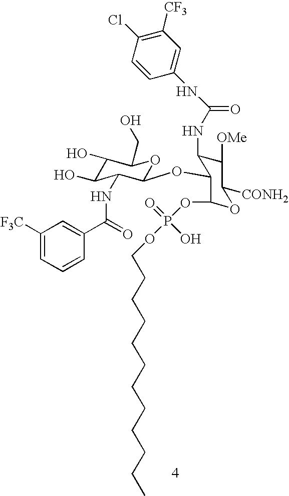

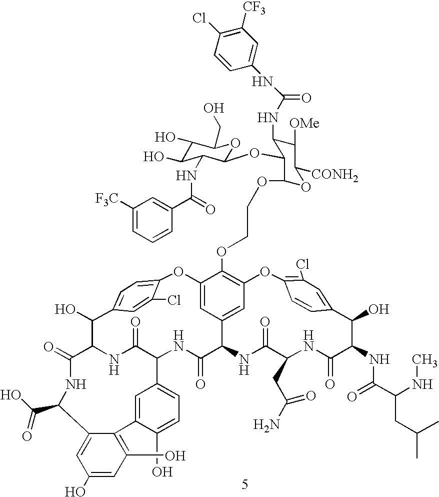

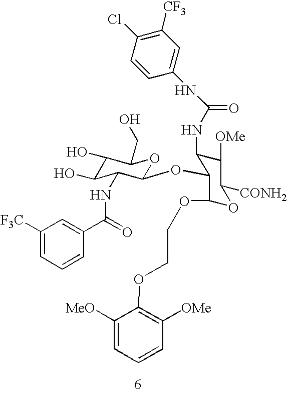

| | E. faecium | | E. faecalis | | |
|---|---|---|---|---|---|
| | | resistant | | resistant | |
| | sensitive | (VanA) | sensitive | (VanB) | S. aureus |
| 4 | 6.2 | 6.2 | 6.2 | 3.1 | 6.2 |
| 5 | 0.1 | 16 | <0.1 | 1 | 0.2 |
| 6 | 250 | 250 | 125 | 250 | 125 |
| Vancomycin | 2 | >500 | 8 | >500 | 2 |
| Aglycon | | | | | |
| 6 + vanco | 1 | 250 | 4 | 250 | 2 |
| aglycone add mix | | | | | |

22-hr MICs (μg/mL)
**Same bacterial strains were used as in Table 1.
^{b}Vancomycin aglycone.
^{c}Add mixture of compound 6 and vancomycin aglycone.

Compound 5 is more effective than compound 2 against resistant strains. In fact, it is comparable to or better against these strains than the glycosidically linked prototype (compound 1b represented in Table 1), while maintaining excellent activity against sensitive strains. Compound 5 is also more active than compound 4, even though it lacks the phospholipid anchor. Compound 6, which does not contain either a phospholipid anchor or the vancomycin aglycone, has less activity. See El-Abadla, N. et al., Tetrahedron, 1999, 55, 699; Sofia, M. et al., J. Med. Chem., 1999, 42, 3193.

The preceding results support the hypothesis that better vancomycin analogs can be made by attaching carbohydrates having good transglycosylase inhibitory activity to the vancomycin aglycone. Compound 5 inhibits transglycosylation in a permeabilized E. coli model, like compound 4 and compounds 1b-3, but unlike vancomycin itself. The functionalized carbohydrate in compound 5 is based on a disaccharide analogue of moenomycin (compound 4), which is a known transglycosylase inhibitor. The phospholipid anchor in compound 4was replaced with the vancomycin aglycone to produce a bifunctional compound. This bifunctional compound has activity that far exceeds the activity of the individual components, as demonstrated by a comparison of the activity of compound 5 to the activity of the mixture of compound 6 with the vancomycin aglycone. The details of this comparison are set forth in Table 2. Of note is the fact that compound5 is more polar than compound 4 or compound 2, both of which contain large hydrophobic groups. Thus, a lipid-based membrane anchor is not essential for overcoming resistant bacteria, which should make it easier to synthesize vancomycin analogs with better physical properties. The synthesis of a large collection of vancomycin analogs will be greatly facilitated by the replacement of the glycosidic linkage with a simple ethylene glycol linker, or any of the coupling moieties discussed above. The bifunctional design concept outlined herein may be expanded to include the many synthetic peptide binders that have been inspired by vancomycin. See Xu, R. et al., J. Am. Chem. Soc., 1999, 121, 4898; Hinzen, B. et al., Helv. Chim. Acta., 1996, 79, 942; Hossain, M. A. et al., J. Am. Chem. Soc., 1998, 120, 11208; Breslow, R. et al., J. Am. Chem. Soc., 1998, 120, 3536; Torneiro, M. et al., Tetrahedron, 1997, 53, 8739; Peczuh et al., J. Am. Chem. Soc., 1997, 119, 9327; Haque, T. S. et al., J. Am. Chem. Soc., 1997, 119, 2303; Nesloney, C. L. et al., J. Am. Chem. Soc., 1996, 118, 5836; Nowick, J. S. et al., J. Am. Chem. Soc., 2001, 123, 5176. The present inventions relate to the design of a large class of new antibiotics comprised of hosts that bind to cell surface peptides attached to specific inhibitors for peptidoglycan-processing enzymes.

Each of the foregoing references is incorporated herein by reference in its entirety. Various modifications of the invention, in addition to those described herein, will be apparent to those skilled in the art. Such modifications are intended to fall within the scope of the appended claims.

What is claimed:

1. An aglycone of vancomycin attached to the anomeric carbon of moenomycin or a moenomycin derivative.

2. The compound of claim 1 having antibiotic activity.

3. The compound of claim 1 wherein said moenomycin derivative is a disaccharide derivative.

4. The compound of claim 1 wherein said moenomycin derivative is a trisaccharide derivative, or a functionalized disaccharide derivative.

5. The compound of claim 1 wherein the aglycone of vancomycin is desleucyl vancomycin aglycone, or N- or C- terminal modified vancomycin aglycone.

6. The compound of claim 1 wherein said anomeric carbon was previously occupied by a phospholipid.

7. The compound of claim 1 wherein said aglycone of vancomycin is attached to said moenomycin or moenomycin derivative via a coupling moiety.

8. The compound of claim 7 wherein said coupling moiety is ethylene glycol.

9. A compound of formula:

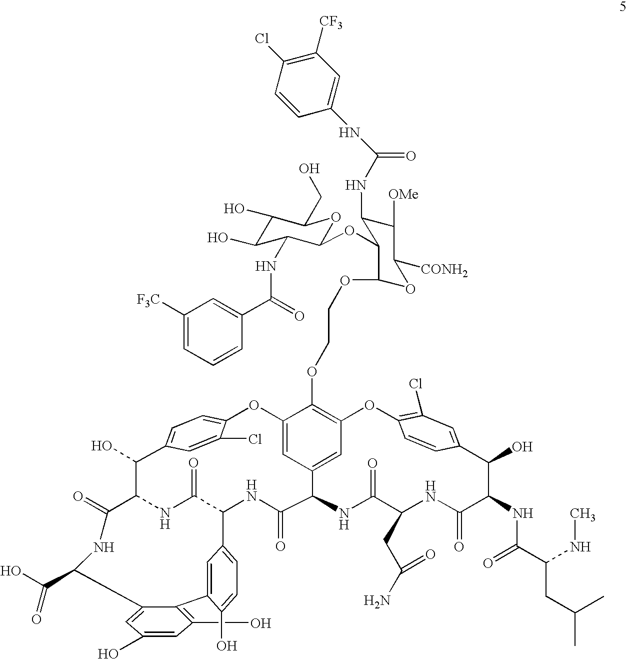

10. A combinatorial library comprising a plurality of moenomycin saccharide derivatives bonded to at least one aglycone of vancomycin.

11. The combinatorial library of claim 10 wherein the phospholipid of the moenomycin derivative has been removed.

12. The combinatorial library of claim 10 wherein said aglycone of vancomycin is desleucyl vancomycin aglycone, or N- or C- terminal modified vancomycin aglycone.

13. The combinatorial library of claim 11 wherein said moenomycin derivative is a disaccharide derivative, a trisaccharide derivative, or a functionalized disaccharide derivative.

14. The combinatorial library of claim 10 wherein one of said plurality of moenomycin derivatives has the formula:

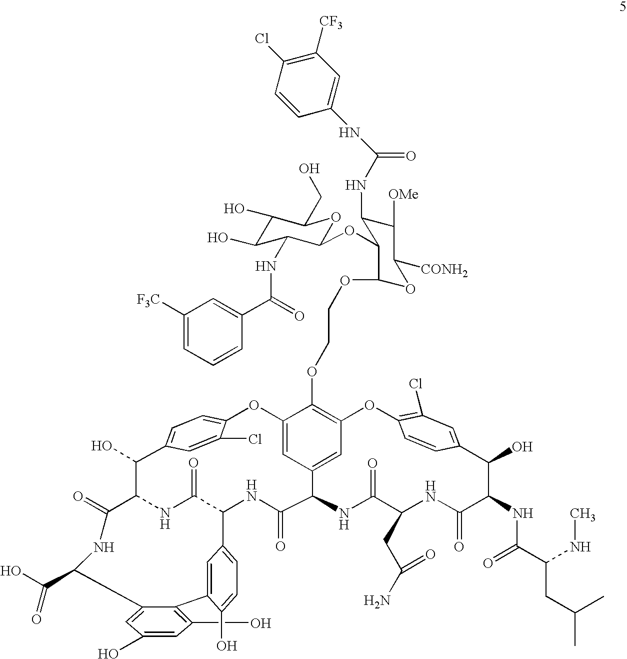

15. A composition comprising a compound comprising an aglycone of vancomycin attached to the anomeric site of moenomycin or a moenomycin derivative.

16. The composition of claim 16 further comprising a pharmaceutically acceptable salt.
